(19) United States
(12) Patent Application Publication
(10) Pub. No.: US 2026/0219348 A1
(43) Pub. Date: Jul. 30, 2026

(54) SYSTEM AND METHOD FOR RF INTERFERENCE REMOVAL FOR OPTIMAL MRI SIGNAL TO INTERFERENCE RATIO

(71) Applicant: GE Precision Healthcare LLC, Waukesha, WI (US)

(72) Inventors: Sreevani Antharjanam Meppallil Illom Venugopal, Bangalore (IN); Harsh Kumar Agarwal, Bangalore (IN); Nitin Jain, Bangalore (IN); Rajagopalan Sundaresan, Bangalore (IN); Ramesh Venkatesan, Bangalore (IN)

(21) Appl. No.: 19/040,247

(22) Filed: Jan. 29, 2025

(51) Int. Cl. G01R 33/565; G01R 33/46; G01R 33/48

(52) U.S. Cl. CPC G01R 33/5659; G01R 33/4625; G01R 33/482

(57) ABSTRACT

A method includes obtaining k-space data of a subject acquired from a plurality of receiving channels of a magnetic resonance imaging scanner during a scan, wherein at least some receiving channels of the plurality of receiving channels are located in different spatial locations from other receiving channels of the plurality of receiving channels. The method includes obtaining channel sensitivity data of the plurality of receiving channels acquired with magnetic resonance imaging scanner prior to acquiring the k-space data. The method includes obtaining noise data for the plurality of receiving channels acquired with magnetic resonance imaging scanner prior to acquiring the k-space data. The method includes linearly combining the k-space data from the plurality of receiving channels based on both the channel sensitivity data and the noise data to estimate virtual channels having virtual k-space data that maximize signal to radio frequency interference ratio while simultaneously isolating zipper artifacts.

### BACKGROUND

The subject matter disclosed herein relates to medical imaging and, more particularly, to a system and a method for radio frequency interference removal for optimal magnetic resonance imaging signal to interference ratio.

Non-invasive imaging technologies allow images of the internal structures or features of a patient/object to be obtained without performing an invasive procedure on the patient/object. In particular, such non-invasive imaging technologies rely on various physical principles (such as the differential transmission of X-rays through a target volume, the reflection of acoustic waves within the volume, the paramagnetic properties of different tissues and materials within the volume, the breakdown of targeted radionuclides within the body, and so forth) to acquire data and to construct images or otherwise represent the observed internal features of the patient/object.

During magnetic resonance imaging (MRI), when a substance such as human tissue is subjected to a uniform magnetic field (polarizing field B₀), the individual magnetic moments of the spins in the tissue attempt to align with this polarizing field, but precess about it in random order at their characteristic Larmor frequency. If the substance, or tissue, is subjected to a magnetic field (excitation field B₁) which is in the x-y plane and which is near the Larmor frequency, the net aligned moment, or “longitudinal magnetization”, M_{z}, may be rotated, or “tipped”, into the x-y plane to produce a net transverse magnetic moment, Mt. A signal is emitted by the excited spins after the excitation signal B₁ is terminated and this signal may be received and processed to form an image.

When utilizing these signals to produce images, magnetic field gradients (Gₓ, G_{y}, and G_{Z}) are employed. Typically, the region to be imaged is scanned by a sequence of measurement cycles in which these gradient fields vary according to the particular localization method being used. The resulting set of received nuclear magnetic resonance (NMR) signals are digitized and processed to reconstruct the image using one of many well-known reconstruction techniques.

An MR image is acquired using coils. Multiple coils and/or multiple channels of one or more coils simultaneously capture signals from different spatial locations. Cartesian k-space acquisition is the most common data acquisition method. In Cartesian space acquisition, a particular direction is called a frequency encoding direction as each spatial location is encoded as a frequency in the MR image acquisition. In the presence of radio frequency interference, the spatial location corresponding to the radio frequency interference frequencies in frequency encoding will be corrupted by the interference signal. If the desired signal and interfering signals occupy the same frequency band, they cannot be effectively separated. Radio frequency manifests as zipper artifacts and high noise levels in MRI and can significantly impact image quality and its diagnostic quality. These zipper artifacts and high noise levels are particularly increased in value MRI systems such as low-field MRI systems and mobile MRI systems due to derated hardware and used case environment.

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a computer-implemented method for removing radio frequency interference is provided. The computer-implemented method includes obtaining, via a processing system including one or more processors, k-space data of a subject acquired from a plurality of receiving channels of a magnetic resonance imaging scanner during a scan, wherein at least some receiving channels of the plurality of receiving channels are located in different spatial locations from other receiving channels of the plurality of receiving channels. The computer-implemented method also includes obtaining, via the processing system, channel sensitivity data of the plurality of receiving channels acquired with magnetic resonance imaging scanner prior to acquiring the k-space data. The computer-implemented method further includes obtaining, via the processing system, noise data for the plurality of receiving channels acquired with magnetic resonance imaging scanner prior to acquiring the k-space data. The computer-implemented method even further includes linearly combining, via the processing system, the k-space data from the plurality of receiving channels based on both the channel sensitivity data and the noise data to estimate virtual channels having virtual k-space data that maximize signal to radio frequency interference ratio while simultaneously isolating zipper artifacts.

In another embodiment, a system for removing radio frequency interference is provided. The system includes a memory encoding processor-executable routines. The system also includes a processing system including one or more processors and configured to access the memory and to execute the processor-executable routines, wherein the process-executable routines, when executed by the processing system, cause the processing system to perform actions. The actions include obtaining k-space data of a subject acquired from a plurality of receiving channels of a magnetic resonance imaging scanner during a scan, wherein at least some receiving channels of the plurality of receiving channels are located in different spatial locations from other receiving channels of the plurality of receiving channels. The actions also include obtaining channel sensitivity data of the plurality of receiving channels acquired with magnetic resonance imaging scanner prior to acquiring the k-space data. The actions further include obtaining noise data for the plurality of receiving channels acquired with magnetic resonance imaging scanner prior to acquiring the k-space data. The actions further include linearly combining the k-space data from the plurality of receiving channels based on both the channel sensitivity data and the noise data to estimate virtual channels having virtual k-space data that maximize signal to radio frequency interference ratio while simultaneously isolating zipper artifacts.

In a further embodiment, a non-transitory computer-readable medium, the computer-readable medium including processor-executable code that when executed by a processing system including one or more processors, causes the processing system to perform actions. The actions include obtaining k-space data of a subject acquired from a plurality of receiving channels of a magnetic resonance imaging scanner during a scan, wherein at least some receiving channels of the plurality of receiving channels are located in different spatial locations from other receiving channels of the plurality of receiving channels. The actions also include obtaining channel sensitivity data of the plurality of receiving channels acquired with magnetic resonance imaging scanner prior to acquiring the k-space data. The actions further include obtaining noise data for the plurality of receiving channels acquired with magnetic resonance imaging scanner prior to acquiring the k-space data. The actions further include linearly combining the k-space data from the plurality of receiving channels based on both the channel sensitivity data and the noise data to estimate virtual channels having virtual k-space data that maximize signal to radio frequency interference ratio while simultaneously isolating zipper artifacts.

### BRIEF DESCRIPTION OF THE DRAWINGS

These and other features, aspects, and advantages of the present subject matter will become better understood when the following detailed description is read with reference to the accompanying drawings in which like characters represent like parts throughout the drawings, wherein:

FIG. 1 depicts a magnetic resonance (MR) image of a portion of a subject having a zipper artifact;

FIG. 2 illustrates a schematic diagram of a magnetic resonance imaging (MRI) system suitable for use with the disclosed techniques;

FIG. 3 is a schematic diagram of an image processing system, in accordance with aspects of the present disclosure;

FIG. 4 is a schematic diagram of a process for removing radio frequency interference and generating zipper free images, in accordance with aspects of the present disclosure;

FIG. 5 depicts images of input receiving channels, in accordance with aspects of the present disclosure;

FIG. 6 depicts images of generated virtual channels, in accordance with aspects of the present disclosure;

FIG. 7 is a flow chart of a method for removing radio frequency interference, in accordance with aspects of the present disclosure;

FIG. 8 depicts images associated with the method for removing radio frequency interference, in accordance with aspects of the present disclosure;

FIG. 9 depicts images from a comparison of the disclosed method to noise whitening, in accordance with aspects of the present disclosure; and

FIG. 10 depicts images from a comparison of the disclosed method to principal component analysis, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

While aspects of the following discussion are provided in the context of medical imaging, it should be appreciated that the disclosed techniques are not limited to such medical contexts. Indeed, the provision of examples and explanations in such a medical context is only to facilitate explanation by providing instances of real-world implementations and applications. However, the disclosed techniques may also be utilized in other contexts, such as image reconstruction for non-destructive inspection of manufactured parts or goods (i.e., quality control or quality review applications), and/or the non-invasive inspection of packages, boxes, luggage, and so forth (i.e., security or screening applications). In general, the disclosed techniques may be useful in any imaging or screening context or image processing or photography field where a set or type of acquired data undergoes a reconstruction process to generate an image or volume.

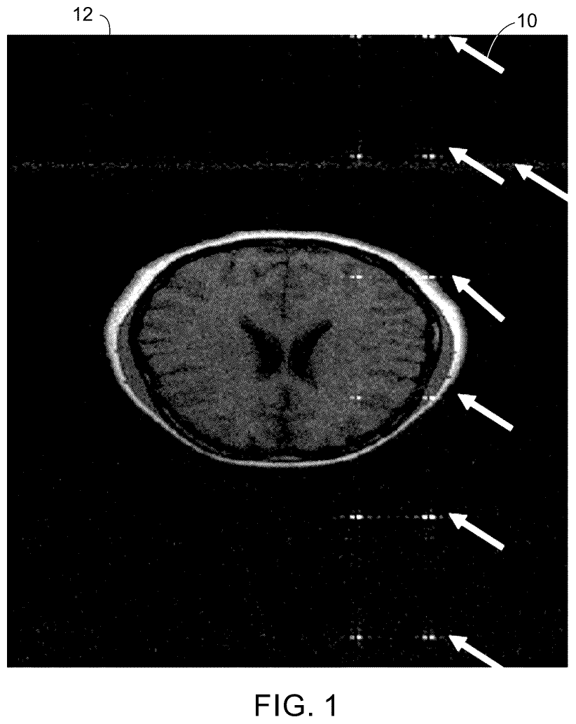

Low cost MRI systems are primarily enabled with deration of hardware such as field strength leading to additional artifacts and image quality degradation. This necessitates compensatory measures within the software reconstruction pipeline to maintain image quality. One of the primary artifacts in these systems is the zipper artifact, which arises due to radio frequency interference arising within or outside the system. A zipper artifact manifests as distinct lines (indicated by arrows 10) along the phase-encoding direction, as depicted in MR image 12 in FIG. 1, and can significantly diminish the diagnostic quality of the resulting images.

The present disclosure provides systems and methods for an MR image reconstruction solution that addresses these issues without requiring additional sensors (e.g., external sensors or coils for detecting radio frequency interference). In particular, the systems and methods are configured to remove radio frequency interferences and, thus, zipper artifacts. The disclosed techniques leverage the redundancy in the radio frequency interference observed across channels in a multi-channel MRI system to detect and to remove zipper artifacts. There is a variation in the desired signal and interference across the channels. All the channels receive radio frequency interferences. However, the variation of the desired signal and radio frequency interference signal across the channels are influenced by factors such as the source of the signal, the orientation and size of a particular channel, and the paths traversed by the signal from the source to a channel. The spatial variation of the desired signal across different channels depends on the spatial sensitivity profile of the receiver coils. This relationship means that the signal changes across channels reflect the inherent sensitivity patterns of the coils/channels, resulting in variations that are specific to each coil's/channel's spatial location and its response to incoming signal. Spatial variation of the desired signal across the channels is smooth and is captured in channel/coil sensitivity matrix while the spatial variation of the radio frequency interference is dependent on the Fourier spectrum of the radio frequency interference. Further, the desired signal source is closer to the receiver channel compared to the radio frequency interference source. These different spatial variations are utilized to linearly combine (e.g., utilizing beamforming) the received signal and then a set of virtual coils/channels are derived that is tailored to the data which effectively separate the radio frequency interference from the desired data simultaneously across the virtual coils/channels. The virtual coils/channels maximize the signal to radio frequency interference ratio. An MR image can be generated whereby the radio frequency interference is separated and suppressed/eliminated by various combinations of the generated virtual channels.

The disclosed techniques provide simultaneous channel compression and artifact removal/noise removal. The disclosed techniques, with the reduction of artifacts, enable radiologists and medical professionals to obtain/utilize higher-quality images which leads to more accurate diagnoses and better patient outcomes. The disclosed techniques provide reduced hardware costs since the need for additional sensors or expensive components is minimized/eliminated. The disclosed techniques provide a software-based solution that is easier to integrate into existing systems, allowing for seamless upgrades and enhancements without significant changes to the hardware.

The disclosed embodiments include a computer-implemented method and a system for removing radio frequency interference that include obtaining, via a processing system including one or more processors, k-space data of a subject acquired from a plurality of receiving channels of a magnetic resonance imaging scanner during a scan, wherein at least some receiving channels of the plurality of receiving channels are located in different spatial locations from other receiving channels of the plurality of receiving channels. The computer-implemented method and systems also include obtaining, via the processing system, channel sensitivity data of the plurality of receiving channels acquired with magnetic resonance imaging scanner prior to acquiring the k-space data. The computer-implemented method and system further include obtaining, via the processing system, noise data for the plurality of receiving channels acquired with magnetic resonance imaging scanner prior to acquiring the k-space data. The computer-implemented method and system even further include linearly combining, via the processing system, the k-space data from the plurality of receiving channels based on both the channel sensitivity data and the noise data to estimate virtual channels having virtual k-space data that maximize signal to radio frequency interference ratio while simultaneously isolating zipper artifacts (e.g., via separation of radio frequency interference from desired signal).

In certain embodiments, the computer-implemented method and system include utilizing, via the processing system, the channel sensitivity data to calculate inter-channel signal correlation matrix. In certain embodiments, the computer-implemented method and system include utilizing, via the processing system, the noise data to calculate inter-channel radio frequency interference correlation matrix. In certain embodiments, the computer-implemented method and system include determining, via the processing system, channel combination weights that maximize signal and minimize radio frequency interference utilizing generalized eigen vector decomposition in calculating the signal to radio frequency interference ratio for each virtual channel based on the inter-channel signal correlation matrix and the inter-channel radio frequency interference correlation matrix. In certain embodiments, estimation of the virtual channels is based on the channel combination weights. In certain embodiments, the computer-implemented method and system include generating, via the processing system, a reconstructed image free of zipper artifacts utilizing the virtual k-space data from a subset of the virtual channels. In certain embodiments, the computer-implemented method and system include determining, via the processing system, the subset of virtual channels to utilize for generating the reconstructed image based on a percentile of a total variance retained from eigenvalues used in estimating each respective virtual channel of the virtual channels.

In certain embodiments, the plurality of receiving channels are part of a plurality of radio frequency coils. In certain embodiments, the plurality of receiving channels are part of a single radio frequency coil. In certain embodiments, the zipper artifacts are removed without utilizing external sensors to determine radio frequency interference.

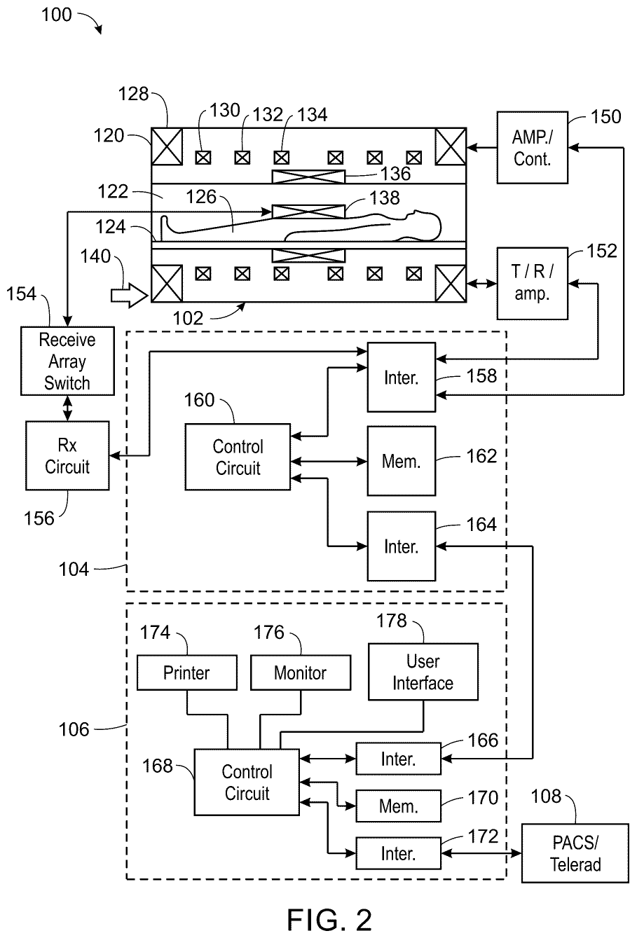

With the preceding in mind, FIG. 2 a magnetic resonance imaging (MRI) system 100 is illustrated schematically as including a scanner 102, scanner control circuitry 104, and system control circuitry 106. According to the embodiments described herein, the MRI system 100 is generally configured to perform MR imaging.

System 100 additionally includes remote access and storage systems or devices such as picture archiving and communication systems (PACS) 108, or other devices such as teleradiology equipment so that data acquired by the system 100 may be accessed on- or off-site. In this way, MR data may be acquired, followed by on- or off-site processing and evaluation. While the MRI system 100 may include any suitable scanner or detector, in the illustrated embodiment, the system 100 includes a full body scanner 102 having a housing 120 through which a bore 122 is formed. A table 124 is moveable into the bore 122 to permit a patient 126 (e.g., subject) to be positioned therein for imaging selected anatomy within the patient.

Scanner 102 includes a series of associated coils for producing controlled magnetic fields for exciting the gyromagnetic material within the anatomy of the patient being imaged. Specifically, a primary magnet coil 128 is provided for generating a primary magnetic field, B₀, which is generally aligned with the bore 122. A series of gradient coils 130, 132, and 134 permit controlled magnetic gradient fields to be generated for positional encoding of certain gyromagnetic nuclei within the patient 126 during examination sequences. A radio frequency (RF) coil 136 (e.g., RF transmit coil) is configured to generate radio frequency pulses for exciting the certain gyromagnetic nuclei within the patient. In addition to the coils that may be local to the scanner 102, the system 100 also includes a set of receiving coils or RF receiving coils 138 (e.g., an array of coils) configured for placement proximal (e.g., against) to the patient 126. As an example, the receiving coils 138 can include cervical/thoracic/lumbar (CTL) coils, head coils, single-sided spine coils, and so forth. Generally, the receiving coils 138 are placed close to or on top of the patient 126 so as to receive the weak RF signals (weak relative to the transmitted pulses generated by the scanner coils) that are generated by certain gyromagnetic nuclei within the patient 126 as they return to their relaxed state.

The various coils of system 100 are controlled by external circuitry to generate the desired field and pulses, and to read emissions from the gyromagnetic material in a controlled manner. In the illustrated embodiment, a main power supply 140 provides power to the primary field coil 128 to generate the primary magnetic field, B₀. A power input (e.g., power from a utility or grid), a power distribution unit (PDU), a power supply (PS), and a driver circuit 150 may together provide power to pulse the gradient field coils 130, 132, and 134. The driver circuit 150 may include amplification and control circuitry for supplying current to the coils as defined by digitized pulse sequences output by the scanner control circuitry 104.

Another control circuit 152 is provided for regulating operation of the RF coil 136. Circuit 152 includes a switching device for alternating between the active and inactive modes of operation, wherein the RF coil 136 transmits and does not transmit signals, respectively. Circuit 152 also includes amplification circuitry configured to generate the RF pulses. Similarly, the receiving coils 138 are connected to switch 154, which is capable of switching the receiving coils 138 between receiving and non-receiving modes. Thus, the receiving coils 138 resonate with the RF signals produced by relaxing gyromagnetic nuclei from within the patient 126 while in the receiving mode, and they do not resonate with RF energy from the transmitting coils (i.e., coil 136) so as to prevent undesirable operation while in the non-receiving mode. Additionally, a receiving circuit 156 is configured to receive the data detected by the receiving coils 138 and may include one or more multiplexing and/or amplification circuits.

It should be noted that while the scanner 102 and the control/amplification circuitry described above are illustrated as being coupled by a single line, many such lines may be present in an actual instantiation. For example, separate lines may be used for control, data communication, power transmission, and so on. Further, suitable hardware may be disposed along each type of line for the proper handling of the data and current/voltage. Indeed, various filters, digitizers, and processors may be disposed between the scanner and either or both of the scanner and system control circuitry 104, 106.

As illustrated, scanner control circuitry 104 includes an interface circuit 158, which outputs signals for driving the gradient field coils and the RF coil and for receiving the data representative of the magnetic resonance signals produced in examination sequences. The interface circuit 158 is coupled to a control and analysis circuit 160. The control and analysis circuit 160 executes the commands for driving the circuit 150 and circuit 152 based on defined protocols selected via system control circuit 106.

Control and analysis circuit 160 also serves to receive the magnetic resonance signals and performs subsequent processing before transmitting the data to system control circuit 106. Scanner control circuit 104 also includes one or more memory circuits 162, which store configuration parameters, pulse sequence descriptions, examination results, and so forth, during operation.

Interface circuit 164 is coupled to the control and analysis circuit 160 for exchanging data between scanner control circuitry 104 and system control circuitry 106. In certain embodiments, the control and analysis circuit 160, while illustrated as a single unit, may include one or more hardware devices. The system control circuit 106 includes an interface circuit 166, which receives data from the scanner control circuitry 104 and transmits data and commands back to the scanner control circuitry 104. The control and analysis circuit 168 may include a CPU in a multi-purpose or application specific computer or workstation. Control and analysis circuit 168 is coupled to a memory circuit 170 to store programming code for operation of the MRI system 100 and to store the processed image data for later reconstruction, display and transmission. The programming code may execute one or more algorithms that, when executed by a processor, are configured to removing radio frequency interference as described below. In certain embodiments, the disclosed techniques may occur on a separate computing device having processing circuitry and memory circuitry.

An additional interface circuit 172 may be provided for exchanging image data, configuration parameters, and so forth with external system components such as remote access and storage devices 108. Finally, the system control and analysis circuit 168 may be communicatively coupled to various peripheral devices for facilitating operator interface and for producing hard copies of the reconstructed images. In the illustrated embodiment, these peripherals include a printer 174, a monitor 176, and user interface 178 including devices such as a keyboard, a mouse, a touchscreen (e.g., integrated with the monitor 176), and so forth.

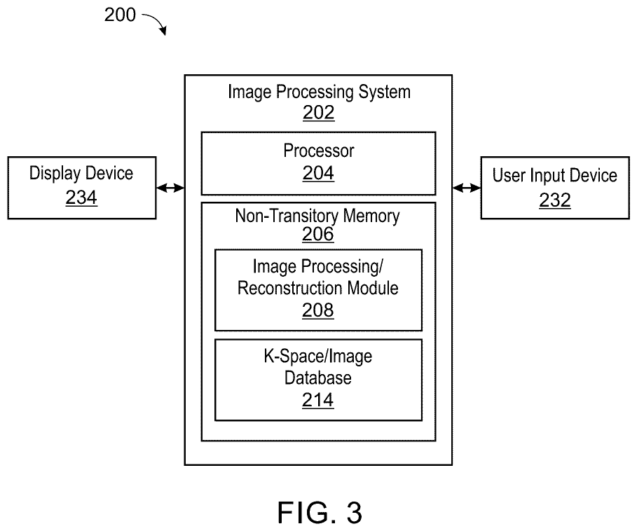

Referring to FIG. 3 an image processing system 202 configured to receive and process k-space data is shown. In some embodiments, the image processing system 202 is incorporated into the MRI system 100 in FIG. 2. For example, the image processing system 202 may be provided in the MRI system 100 as data processing unit. In some embodiments, at least a portion of image processing system 202 is disposed at a device (e.g., edge device, server, etc.) communicably coupled to the MRI system 100 via wired and/or wireless connections. In some embodiments, at least a portion of image processing system 202 is disposed at a separate device (e.g., a workstation) which can receive k-space data from the MRI system 100 or from a storage device which stores the images/k-space data generated by the MRI system 100. The image processing system 202 may be operably/communicatively coupled to a user input device 232 and a display device 234. User input device 232 may be integrated into an MRI system, such as at user input device of the MRI system 100. Similarly, display device 234 may be integrated into an MRI system, such as at display device of MRI system 100.

The image processing system 202 includes a processor 204 configured to execute machine readable instructions stored in non-transitory memory 206. The processor 204 may be single core or multi-core, and the programs executed thereon may be configured for parallel or distributed processing. In some embodiments, the processor 204 may optionally include individual components that are distributed throughout two or more devices, which may be remotely located and/or configured for coordinated processing. In some embodiments, one or more aspects of processor 204 may be virtualized and executed by remotely-accessible networked computing devices configured in a cloud computing configuration.

Then non-transitory memory 206 may store an image processing/reconstruction module 208 and a k-space/image database 214. The image processing/reconstruction module 208 may obtain the k-space data from the k-space image database. The image processing/reconstruction module 208 is configured to remove radio frequency interference. The image processing/reconstruction module 208 is configured to obtain k-space data of a subject acquired from a plurality of receiving channels (of one or more coils) of a magnetic resonance imaging scanner during a scan, wherein at least some receiving channels of the plurality of receiving channels are located in different spatial locations from other receiving channels of the plurality of receiving channels. The image processing/reconstruction module 208 is configured to obtain channel sensitivity data of the plurality of receiving channels acquired with magnetic resonance imaging scanner prior to acquiring the k-space data. The image processing/reconstruction module 208 is configured to obtain noise data for the plurality of receiving channels acquired with magnetic resonance imaging scanner prior to acquiring the k-space data. The image processing/reconstruction module 208 is configured to linearly combine the k-space data from the plurality of receiving channels based on both the channel sensitivity data and the noise data to estimate virtual channels having virtual k-space data that maximize signal to radio frequency interference ratio while simultaneously isolating zipper artifacts (e.g., via separation of radio frequency interference from desired signal). Optimizing the signal to radio frequency interference avoids signal loss that occurs if minimizing radio interference alone.

In certain embodiments, the image processing/reconstruction module 208 is configured to utilize the channel sensitivity data to calculate inter-channel signal correlation matrix. In certain embodiments, the image processing/reconstruction module 208 is configured to utilize the noise data to calculate inter-channel radio frequency interference correlation matrix. In certain embodiments, image processing/reconstruction module 208 is configured to determine channel combination weights that maximize signal and minimize radio frequency interference utilizing generalized eigen vector decomposition in calculating the signal to radio frequency interference ratio for each virtual channel based on the inter-channel signal correlation matrix and the inter-channel radio frequency interference correlation matrix. In certain embodiments, estimation of the virtual channels is based on the channel combination weights. In certain embodiments, the image processing/reconstruction module 208 is configured to generate a reconstructed image free of zipper artifacts utilizing the virtual k-space data from a subset of the virtual channels. In certain embodiments, the image processing/reconstruction module 208 is configured to determine the subset of virtual channels to utilize for generating the reconstructed image based on a percentile of a total variance retained from eigenvalues used in estimating each respective virtual channel of the virtual channels.

In certain embodiments, the plurality of receiving channels are part of a plurality of radio frequency coils. In certain embodiments, the plurality of receiving channels are part of a single radio frequency coil. In certain embodiments, the zipper artifacts are removed without utilizing external sensors to determine radio frequency interference.

Non-transitory memory 206 further stores k-space/image database 214. The k-space/image database 214 may include, for example, k-space data acquired via an MRI system and images reconstructed from the k-space data. For example, k-space/image database 214 may store k-space data acquired via MRI system 100, and/or received from other communicatively coupled MRI systems or image databases. In some examples, k-space/image database 214 may store images reconstructed by the image processing/reconstruction module 208.

In some embodiments, non-transitory memory 206 may include components disposed at two or more devices, which may be remotely located and/or configured for coordinated processing. In some embodiments, one or more aspects of non-transitory memory 206 may include remotely-accessible networked storage devices configured in a cloud computing configuration.

User input device 232 may include one or more of a touchscreen, a keyboard, a mouse, a trackpad, a motion sensing camera, or other device configured to enable a user to interact with and manipulate data within image processing system 202. Display device 234 may include one or more display devices utilizing virtually any type of technology. In some embodiments, display device 234 may comprise a computer monitor, and may display MR images. Display device 234 may be combined with processor 204, non-transitory memory 206, and/or user input device 232 in a shared enclosure, or may be peripheral display devices and may comprise a monitor, touchscreen, projector, or other display device known in the art, which may enable a user to view MRI images produced by an MRI system, and/or interact with various data stored in non-transitory memory 206.

It should be understood that image processing system 202 shown in FIG. 3 is for illustration, not for limitation. Another appropriate image processing system may include more, fewer, or different components.

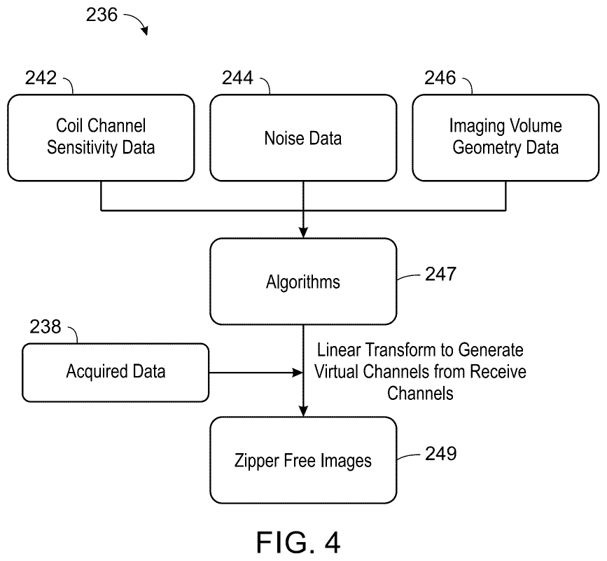

FIG. 4 is a schematic diagram of a process 236 for removing radio frequency interference (while optimizing signal to interference ratio) and generating zipper free images. The process 236 includes, in the presence of stationary radio frequency interference (i.e., not changing between noise data and imaging data acquisitions), estimating virtual channels using the channel/coil sensitivity matrix and noise that was acquired prior the imaging data acquisition (i.e., acquisition of data of a subject). The process 236 includes acquiring data as indicated by reference numeral 238. The data is k-space data acquired of a subject from multiple channels (e.g., receiving channels of one or more receiving radio frequency coils such as body coils or surface coils) at different spatial locations of a magnetic resonance imaging scanner. Let cᵢ be the acquired k-space signal in the i^{th} receiver channel. It can be modeled by the following:

$${c}_{i} ( k ) = {s}_{i} ( k ) + {n}_{i} ( k ) + {r}_{i} ( k ) \qquad ( 1 )$$

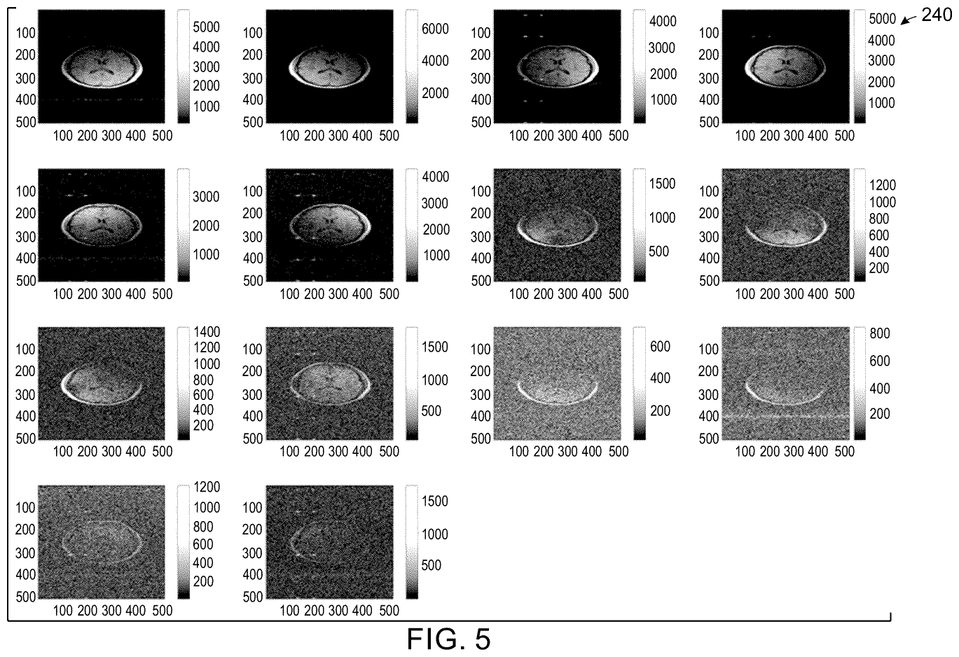

for i=1, 2, . . . . N_{c}, where N_{c} is the number of channels, sᵢ represents the signal, and nᵢ represents the noise, and rᵢ represents the radio interference. FIG. 5 depicts the images 240 of signals simultaneously captured by a plurality of receiving channels (e.g., 14 input channels) at different spatial locations.

The process 236 also includes obtaining coil/channel sensitivity data (e.g., coil/channel sensitivity maps) as part of the imaging session (e.g., during a prescan) (but prior to acquiring the imaging data (i.e., acquired k-space data 238) over the imaging volume of the subject as indicated by reference numeral 242. The coil/channel sensitivity maps are utilized to obtain inter-coil/channel signal correlation matrix, R_{S}. The inter-coil/channel signal correlation matrix, R_{S}, is a cross correlation of the coil/channel sensitivity vector across the slice is represented in the following: R_{S}=E (S S^{H}). The data from each channel is transformed into a column, forming the matrix that serves as the basis for the analysis. The advantage of using sensitivity maps over direct imaging data is that it provides a more accurate approximation of noise/artifact-free signal.

The process 236 also includes obtaining noise data as part of the imaging session (e.g., during a prescan) (but prior to acquiring the imaging data (i.e., acquired k-space data 238) without exciting any of the imaging volume as indicated by reference numeral 244. The noise data is utilized to obtain/estimate an inter-coil/channel radio frequency interference correlation matrix, R_{Z}. The inter-coil/channel signal radio frequency interference correlation matrix, R_{Z}, is a cross correlation of the noise vector across a desired frequency range of radio frequency interference is represented in the following: R_{Z}=E (R R^{H}). In certain embodiments, for a more precise approximation of zipper variance, peaks in the frequency spectrum of the noise data are identified. A range of values around these peaks were selected to calculate the radio frequency interference correlation matrix. In certain embodiments, the process 236 also includes obtaining imaging volume geometry data as part of the imaging session (e.g., from calibration during a prescan) (but prior to acquiring the imaging data (i.e., acquired k-space data 238) as indicated by reference numeral 246.

As noted above, different spatial variations are utilized to linearly combine (e.g., utilizing beamforming) the received signal and then a set of virtual coils/channels are derived that is tailored to the data which effectively separate the radio frequency interference from the desired data simultaneously across the virtual coils/channels. The process 236 enables simultaneous performance of channel compression and radio frequency interference/zipper artifact removal. In particular, one or more algorithms (as indicated by reference numeral 247) to remove radio frequency interference (and zipper artifacts) to maximize MRI signal to interference ratio. The algorithms linearly combine (e.g., utilizing beam transforming) the k-space data from the plurality of receiving channels (via compression of the receiving channels) based on both the channel sensitivity data and the noise data to estimate virtual channels having virtual k-space data that maximize signal to radio frequency interference ratio while simultaneously isolating zipper artifacts (e.g., via separation of radio frequency interference from desired signal). Optimizing the signal to radio frequency interference avoids signal loss that occurs if minimizing radio interference alone. In certain embodiments, the imaging volume geometry data may also be utilized in estimating the virtual channels.

The signal to interference ratio in a virtual channel i with channel combination weights wᵢ is given by:

$$SIR = \frac{{W}_{i}^{H} {R}_{S} W}{{W}_{i}^{H} {R}_{Z} {W}_{i}} . \qquad ( 2 )$$

The channel combination weights that maximize signal and minimizes radio frequency interference or increase the separation between the signal and interference can be found using the signal to interference (zipper or radio frequency interference) ratio (SIR). Equation 4 has the form of a generalized Rayleigh quotient. The channel combination weights to generate the virtual channels are in the order of SIR and that are ortho-normal across the channels can be generated using generalized eigen decomposition as a standard eigenvalue problem for the matrix

$${R}_{Z}^{- 1} {R}_{S} .$$

In particular, the ordered eigenvectors, W, generate virtual coils/channels with ordered values of SIR. In certain embodiments, Tikhonov regularization can be applied to improve the stability of the R_{Z}⁻¹ estimation. Upon generating the channel combination weights, the final matrix (i.e., final form of Equation 4) is utilized in channel compression as a weight basis matrix.

The acquired data Cᵢ can be linearly transformed to virtual channel data according to:

$${v}_{j} ( k ) = {\sum }_{i = 1}^{{N}_{c}} {w}_{ij} {c}_{i} ( k ) \qquad ( 3 )$$

for j=1, 2, . . . . N_{c}, where N_{c} is the number of channels, wᵢⱼ represent the channel combination weights, and vⱼ(k) represents the j^{th} virtual channel. This can be expressed in matrix form as:

$$V = {W}^{H} C \qquad ( 4 )$$

where V, W, and C are the matrices formed by stacking the vectors vᵢ, wᵢ, and cᵢ as column vectors, respectively.

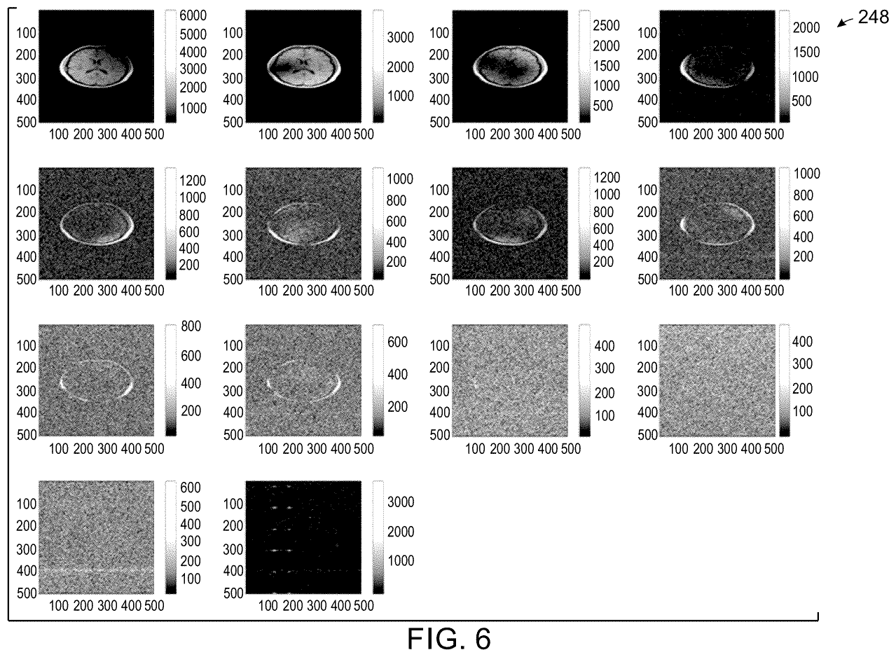

FIG. 6 depicts images 248 of generated virtual channels (in order of greatest eigenvalue to least eigenvalue). Virtual channels corresponding to the largest eigenvalue maximizes the signal while minimizing the interference, effectively enhancing the desired signal. Conversely, virtual channels corresponding to the smallest eigenvalue minimizes the signal and maximizes the interference. In certain embodiments, the virtual channels can be derived prior to scan for acquiring the imaging data. The process 236 includes generating a reconstructed image 249 (e.g., zipper free image) utilizing the virtual k-space data from a subset of the virtual channels. The number (i.e., subset) of the virtual channels is based on a percentile of a total variance retained from eigenvalues used in estimating each respective virtual channel of the virtual channels. This ensures that only the most significant components are used. In certain embodiments, other virtual channel combination schemes may be utilized to improve the SIR.

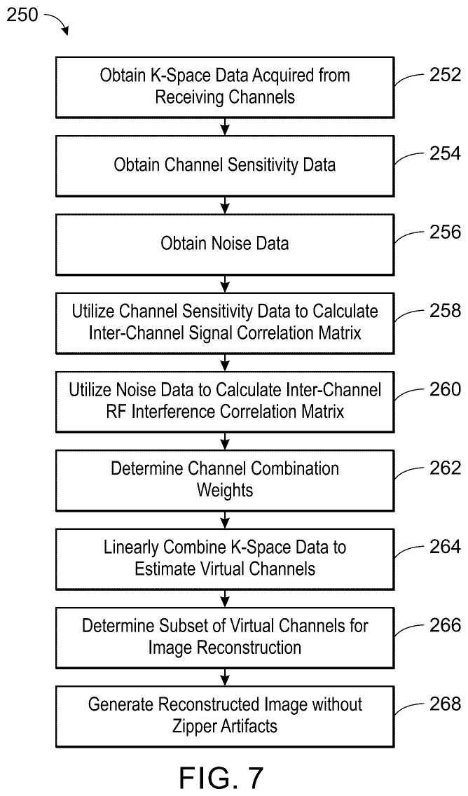

FIG. 7 illustrates a flow diagram of a method 250 removing radio frequency interference (while optimizing signal to interference ratio). One or more steps of the method 1100 may be performed by processing circuitry of the magnetic resonance imaging system 100 in FIG. 2 or a remote computing device.

The method 250 includes obtaining k-space data of a subject acquired from a plurality of receiving channels of a magnetic resonance imaging scanner during a scan (block 252). At least some receiving channels of the plurality of receiving channels are located in different spatial locations from other receiving channels of the plurality of receiving channels. In certain embodiments, some of the receiving channels may overlap. In certain embodiments, each of the receiving channels may be if different spatial locations. In certain embodiments, the plurality of receiving channels are part of a plurality of radio frequency coils. In certain embodiments, the plurality of receiving channels are part of a single radio frequency coil.

The method 250 also includes obtaining channel sensitivity data (e.g., channel sensitivity maps) (over the imaging volume of the subject) of the plurality of receiving channels acquired with magnetic resonance imaging scanner prior to acquiring the k-space data (block 254). The channel sensitivity data is acquired as part of the imaging session (e.g., during a prescan) but prior to acquiring the imaging data.

The method 250 further includes obtaining noise data for the plurality of receiving channels acquired with magnetic resonance imaging scanner prior to acquiring the k-space data (block 256). The noise data is acquired (without exciting any of the imaging volume) as part of the imaging session (e.g., during a prescan) but prior to acquiring the imaging data.

The method 250 even further includes utilizing the channel sensitivity data to calculate the inter-channel signal correlation matrix (R_{S}) (block 258). The data from each channel is transformed into a column, forming the matrix that serves as the basis for the analysis. The advantage of using sensitivity maps over direct imaging data is that it provides a more accurate approximation of noise/artifact-free signal.

The method 250 still further includes utilizing the noise data to calculate the inter-channel radio frequency interference correlation matrix (R_{Z}) (block 260). In certain embodiments, for a more precise approximation of zipper variance, peaks in the frequency spectrum of the noise data are identified. A range of values around these peaks were selected to calculate the radio frequency interference correlation matrix.

The method 250 yet further includes determining channel combination weights that maximize signal and minimize radio frequency interference utilizing generalized eigen vector decomposition in calculating the signal to radio frequency interference ratio for each virtual channel based on the inter-channel signal correlation matrix and the inter-channel radio frequency interference correlation matrix (block 262). The channel combination weights may be determined using Equation 4. The method 250 further includes linearly combining (e.g. compressing) the k-space data from the plurality of receiving channels based on both the channel sensitivity data and the noise data to estimate virtual channels having virtual k-space data that maximize signal to radio frequency interference ratio while simultaneously isolating zipper artifacts (e.g., via separation of radio frequency interference from desired signal) (block 264). Estimation of the virtual channels is based on the channel combination weights. Equation 3 may be utilized to estimate the virtual channels.

In certain embodiments, the method 250 includes determining the subset of virtual channels to utilize for generating the reconstructed image based on a percentile of a total variance retained from eigenvalues used in estimating each respective virtual channel of the virtual channels (block 266). The method 250 includes generating a reconstructed image utilizing the virtual k-space data from a subset (i.e., less than the total of all the virtual channels) of the virtual channels (block 268). This ensures that only the most significant components are used. In certain embodiments, other virtual channel combination schemes may be utilized to improve the SIR. The reconstructed image is free of zipper artifacts.

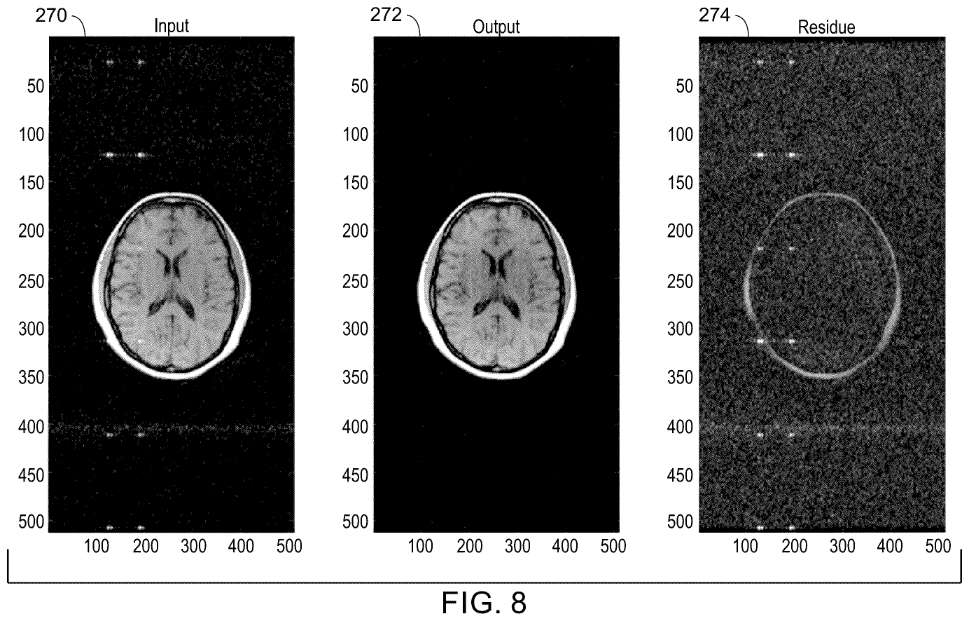

FIG. 8 depicts images associated with the method (e.g., method 250 in FIG. 7) for removing radio frequency interference. Image 270 is an input MR image of a portion (e.g., brain) of a subject. Image 270 is a channel-combined image of the input channels (i.e., receiving channels of one or more coils) that is utilized with the algorithms discussed above. Image 272 is an output MR image utilizing the method 250 to remove radio frequency interference. The image 272 is a reconstructed image from a subset of estimated virtual channels. Image 272 is free of zipper artifacts. Image 274 is of the residual data discarded utilizing the method 250.

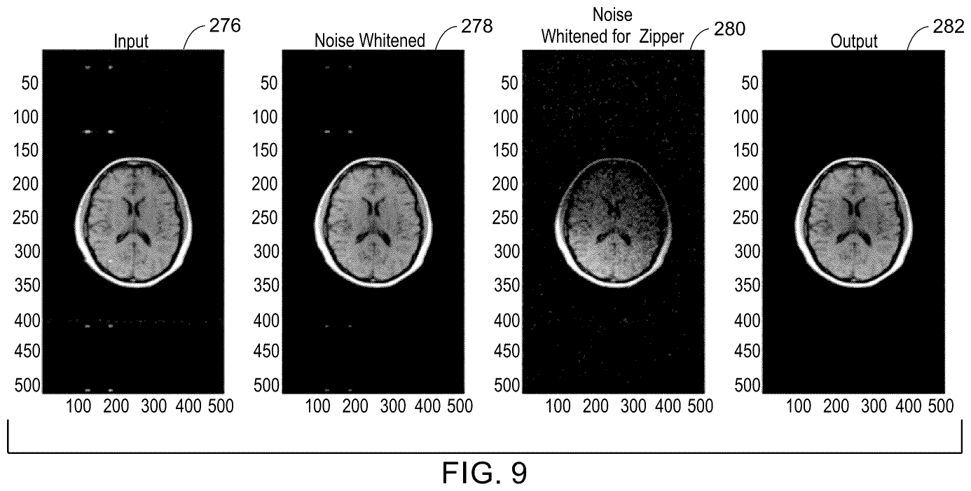

FIG. 9 depicts images from a comparison of the disclosed method to noise whitening. Image 276 is an input MR image of a portion (e.g., brain) of a subject. Image 276 is a channel-combined image of the input channels (i.e., receiving channels of one or more coils) that is utilized with the different techniques for channel compression and noise removal. Image 278 is an output MR image generated utilizing standard noise whitening. Image 280 is an output MR image generated utilizing tailored noise whitening for zipper artifact removal. Image 282 is an output MR image generated utilizing the method 250 in FIG. 7. As depicted, the standard noise whitening technique fails to completely remove the zipper artifacts, leaving residual distortions in the image 278. On the other hand, with the tailored noise whitening technique effectively removes zipper artifacts, it does so at the expense of image quality, leading to noticeable degradation as depicted in image 280. This trade-off highlights the challenges of achieving both artifact removal and image preservation simultaneously. Image 282 has the zipper artifacts removed while providing a high-quality image.

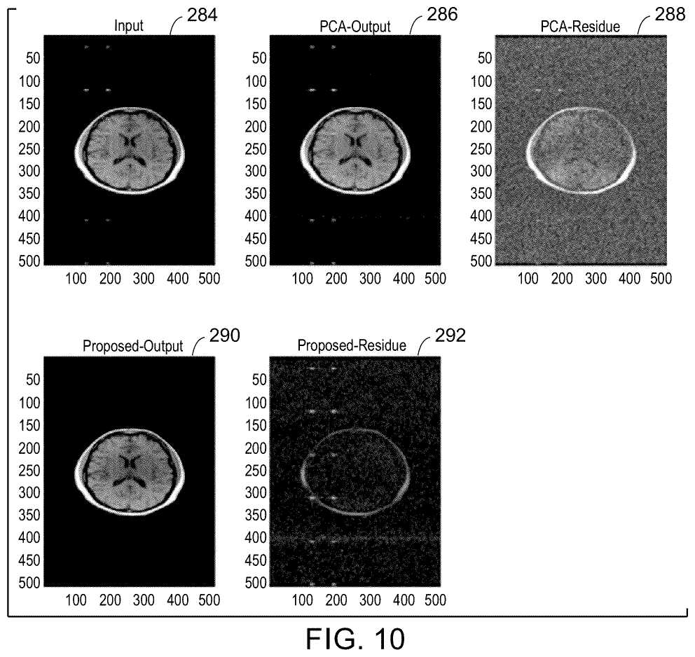

FIG. 10 depicts images from a comparison of the disclosed method to principal component analysis (PCA). Image 284 is an input MR image of a portion (e.g., brain) of a subject. Image 284 is a channel-combined image of the input channels (i.e., receiving channels of one or more coils) that is utilized with the different techniques. Principal component analysis is the most used channel compression technique. Image 286 is an output MR image generated utilizing principal component analysis. Image 288 is of the residual data discarded utilizing principal component analysis. Image 290 is an output MR image generated utilizing the method 250 in FIG. 7. Image 292 is of the residual data discarded utilizing the method 250 in FIG. 7. As depicted, principal component analysis fails to remove zipper artifacts and even loses more image information compared to the method 250.

Technical effects of the disclosed subject matter include providing simultaneous channel compression and artifact removal/noise removal. Technical effects of the disclosed subject matter include, with the reduction of artifacts, enabling radiologists and medical professionals to obtain/utilize higher-quality images which leads to more accurate diagnoses and better patient outcomes. Technical effects of the disclosed subject matter include providing reduced hardware costs since the need for additional sensors or expensive components is minimized/eliminated. Technical effects of the disclosed subject matter include providing a software-based solution that is easier to integrate into existing systems, allowing for seamless upgrades and enhancements without significant changes to the hardware.

The disclosure also provides support for a computer-implemented method for removing radio frequency interference, comprising: obtaining, via a processing system comprising one or more processors, k-space data of a subject acquired from a plurality of receiving channels of a magnetic resonance imaging scanner during a scan, wherein at least some receiving channels of the plurality of receiving channels are located in different spatial locations from other receiving channels of the plurality of receiving channels; obtaining, via the processing system, channel sensitivity data of the plurality of receiving channels acquired with magnetic resonance imaging scanner prior to acquiring the k-space data; obtaining, via the processing system, noise data for the plurality of receiving channels acquired with magnetic resonance imaging scanner prior to acquiring the k-space data; and linearly combining, via the processing system, the k-space data from the plurality of receiving channels based on both the channel sensitivity data and the noise data to estimate virtual channels having virtual k-space data that maximize signal to radio frequency interference ratio while simultaneously isolating zipper artifacts. In a first example of the computer-implemented method, the computer-implemented method further comprises utilizing, via the processing system, the channel sensitivity data to calculate inter-channel signal correlation matrix. In a second example of the computer-implemented method, optionally including the first example, the computer-implemented method further comprises utilizing, via the processing system, the noise data to calculate inter-channel radio frequency interference correlation matrix. In a third example of the computer-implemented method, optionally including one or both of the first and second examples, the computer-implemented method further comprises determining, via the processing system, channel combination weights that maximize signal and minimize radio frequency interference utilizing generalized eigen vector decomposition in calculating the signal to radio frequency interference ratio for each virtual channel based on the inter-channel signal correlation matrix and the inter-channel radio frequency interference correlation matrix. In a fourth example of the computer-implemented method, optionally including one or more or each of the first through third examples, estimation of the virtual channels is based on the channel combination weights. In a fifth example of the computer-implemented method, optionally including one or more or each of the first through fourth examples, the computer-implemented method further comprises generating, via the processing system, a reconstructed image free of zipper artifacts utilizing the virtual k-space data from a subset of the virtual channels. In a sixth example of the computer-implemented method, optionally including one or more or each of the first through fifth examples, the computer-implemented method further comprises determining, via the processing system, the subset of virtual channels to utilize for generating the reconstructed image based on a percentile of a total variance retained from eigenvalues used in estimating each respective virtual channel of the virtual channels. In a seventh example of the computer-implemented method, optionally including one or more or each of the first through sixth examples, the zipper artifacts are removed without utilizing external sensors to determine the radio frequency interference. In an eighth example of the computer-implemented method, optionally including one or more or each of the first through seventh examples, the plurality of receiving channels are part of a plurality of radio frequency coils. In a ninth example of the computer-implemented method, optionally including one or more or each of the first through eighth examples, the plurality of receiving channels are part of a single radio frequency coil.

The disclosure also provides support for a system for removing radio frequency interference, comprising: a memory encoding processor-executable routines; and a processing system comprising one or more processors and configured to access the memory and to execute the processor-executable routines, wherein the processor-executable routines, when executed by the processing system, cause the processing system to: obtain k-space data of a subject acquired from a plurality of receiving channels of a magnetic resonance imaging scanner during a scan, wherein at least some receiving channels of the plurality of receiving channels are located in different spatial locations from other receiving channels of the plurality of receiving channels; obtain channel sensitivity data of the plurality of receiving channels acquired with magnetic resonance imaging scanner prior to acquiring the k-space data; obtain noise data for the plurality of receiving channels acquired with magnetic resonance imaging scanner prior to acquiring the k-space data; and linearly combine the k-space data from the plurality of receiving channels based on both the channel sensitivity data and the noise data to estimate virtual channels having virtual k-space data that maximize signal to radio frequency interference ratio while simultaneously isolating zipper artifacts. In a first example of the system, the processor-executable routines, when executed by the processing system, further cause the processing system to utilize the channel sensitivity data to calculate inter-channel signal correlation matrix. In a second example of the system, optionally including the first example, the processor-executable routines, when executed by the processing system, further cause the processing system to utilize the noise data to calculate inter-channel radio frequency interference correlation matrix. In a third example of the system, optionally including one or both of the first and second examples, the processor-executable routines, when executed by the processing system, further cause the processing system to determine channel combination weights that maximize signal and minimize radio frequency interference utilizing generalized eigen vector decomposition in calculating the signal to radio frequency interference ratio for each virtual channel based on the inter-channel signal correlation matrix and the inter-channel radio frequency interference correlation matrix. In a fourth example of the system, optionally including one or more or each of the first through third examples, estimation of the virtual channels is based on the channel combination weights. In a fifth example of the system, optionally including one or more or each of the first through fourth examples, the processor-executable routines, when executed by the processing system, further cause the processing system to generate a reconstructed image free of zipper artifacts utilizing the virtual k-space data from a subset of the virtual channels. In a sixth example of the system, optionally including one or more or each of the first through fifth examples, the processor-executable routines, when executed by the processing system, further cause the processing system to determine the subset of virtual channels to utilize for generating the reconstructed image based on a percentile of a total variance retained from eigenvalues used in estimating each respective virtual channel of the virtual channels. In a seventh example of the system, optionally including one or more or each of the first through sixth examples, the zipper artifacts are removed without utilizing external sensors to determine the radio frequency interference.

The disclosure also provides support for a non-transitory computer-readable medium, the computer-readable medium comprising processor-executable code that when executed by a processing system comprising one or more processors, causes the processing system to: obtain k-space data of a subject acquired from a plurality of receiving channels of a magnetic resonance imaging scanner during a scan, wherein at least some receiving channels of the plurality of receiving channels are located in different spatial locations from other receiving channels of the plurality of receiving channels; obtain channel sensitivity data of the plurality of receiving channels acquired with magnetic resonance imaging scanner prior to acquiring the k-space data; obtain noise data for the plurality of receiving channels acquired with magnetic resonance imaging scanner prior to acquiring the k-space data; and linearly combine the k-space data from the plurality of receiving channels based on both the channel sensitivity data and the noise data to estimate virtual channels having virtual k-space data that maximize signal to radio frequency interference ratio while simultaneously isolating zipper artifacts. In a first example of the non-transitory computer-readable medium, the processor-executable code, when executed by the processing system, further causes the processing system to generate a reconstructed image free of zipper artifacts utilizing the virtual k-space data from a subset of the virtual channels.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform] ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A computer-implemented method for removing radio frequency interference, comprising:
obtaining, via a processing system comprising one or more processors, k-space data of a subject acquired from a plurality of receiving channels of a magnetic resonance imaging scanner during a scan, wherein at least some receiving channels of the plurality of receiving channels are located in different spatial locations from other receiving channels of the plurality of receiving channels;
obtaining, via the processing system, channel sensitivity data of the plurality of receiving channels acquired with magnetic resonance imaging scanner prior to acquiring the k-space data;
obtaining, via the processing system, noise data for the plurality of receiving channels acquired with magnetic resonance imaging scanner prior to acquiring the k-space data; and
linearly combining, via the processing system, the k-space data from the plurality of receiving channels based on both the channel sensitivity data and the noise data to estimate virtual channels having virtual k-space data that maximize signal to radio frequency interference ratio while simultaneously isolating zipper artifacts.

2. The computer-implemented method of claim 1, further comprising utilizing, via the processing system, the channel sensitivity data to calculate inter-channel signal correlation matrix.

3. The computer-implemented method of claim 2, further comprising utilizing, via the processing system, the noise data to calculate inter-channel radio frequency interference correlation matrix.

4. The computer-implemented method of claim 3, further comprising determining, via the processing system, channel combination weights that maximize signal and minimize radio frequency interference utilizing generalized eigen vector decomposition in calculating the signal to radio frequency interference ratio for each virtual channel based on the inter-channel signal correlation matrix and the inter-channel radio frequency interference correlation matrix.

5. The computer-implemented method of claim 4, wherein estimation of the virtual channels is based on the channel combination weights.

6. The computer-implemented method of claim 5, further comprising generating, via the processing system, a reconstructed image free of zipper artifacts utilizing the virtual k-space data from a subset of the virtual channels.

7. The computer-implemented method of claim 6, further comprising determining, via the processing system, the subset of virtual channels to utilize for generating the reconstructed image based on a percentile of a total variance retained from eigenvalues used in estimating each respective virtual channel of the virtual channels.

8. The computer-implemented method of claim 5, wherein the zipper artifacts are removed without utilizing external sensors to determine the radio frequency interference.

9. The computer-implemented method of claim 1, wherein the plurality of receiving channels are part of a plurality of radio frequency coils.

10. The computer-implemented method of claim 1, wherein the plurality of receiving channels are part of a single radio frequency coil.

11. A system for removing radio frequency interference, comprising:
a memory encoding processor-executable routines; and
a processing system comprising one or more processors and configured to access the memory and to execute the processor-executable routines, wherein the processor-executable routines, when executed by the processing system, cause the processing system to:
obtain k-space data of a subject acquired from a plurality of receiving channels of a magnetic resonance imaging scanner during a scan, wherein at least some receiving channels of the plurality of receiving channels are located in different spatial locations from other receiving channels of the plurality of receiving channels;
obtain channel sensitivity data of the plurality of receiving channels acquired with magnetic resonance imaging scanner prior to acquiring the k-space data;
obtain noise data for the plurality of receiving channels acquired with magnetic resonance imaging scanner prior to acquiring the k-space data; and
linearly combine the k-space data from the plurality of receiving channels based on both the channel sensitivity data and the noise data to estimate virtual channels having virtual k-space data that maximize signal to radio frequency interference ratio while simultaneously isolating zipper artifacts.

12. The system of claim 11, wherein the processor-executable routines, when executed by the processing system, further cause the processing system to utilize the channel sensitivity data to calculate inter-channel signal correlation matrix.

13. The system of claim 12, wherein the processor-executable routines, when executed by the processing system, further cause the processing system to utilize the noise data to calculate inter-channel radio frequency interference correlation matrix.

14. The system of claim 13, wherein the processor-executable routines, when executed by the processing system, further cause the processing system to determine channel combination weights that maximize signal and minimize radio frequency interference utilizing generalized eigen vector decomposition in calculating the signal to radio frequency interference ratio for each virtual channel based on the inter-channel signal correlation matrix and the inter-channel radio frequency interference correlation matrix.

15. The system of claim 14, wherein estimation of the virtual channels is based on the channel combination weights.

16. The system of claim 15, wherein the processor-executable routines, when executed by the processing system, further cause the processing system to generate a reconstructed image free of zipper artifacts utilizing the virtual k-space data from a subset of the virtual channels.

17. The system of claim 16, wherein the processor-executable routines, when executed by the processing system, further cause the processing system to determine the subset of virtual channels to utilize for generating the reconstructed image based on a percentile of a total variance retained from eigenvalues used in estimating each respective virtual channel of the virtual channels.

18. The system of claim 16, wherein the zipper artifacts are removed without utilizing external sensors to determine radio frequency interference.

19. A non-transitory computer-readable medium, the non-transitory computer-readable medium comprising processor-executable code that when executed by a processing system comprising one or more processors, causes the processing system to:
obtain k-space data of a subject acquired from a plurality of receiving channels of a magnetic resonance imaging scanner during a scan, wherein at least some receiving channels of the plurality of receiving channels are located in different spatial locations from other receiving channels of the plurality of receiving channels;
obtain channel sensitivity data of the plurality of receiving channels acquired with magnetic resonance imaging scanner prior to acquiring the k-space data;
obtain noise data for the plurality of receiving channels acquired with magnetic resonance imaging scanner prior to acquiring the k-space data; and
linearly combine the k-space data from the plurality of receiving channels based on both the channel sensitivity data and the noise data to estimate virtual channels having virtual k-space data that maximize signal to radio frequency interference ratio while simultaneously isolating zipper artifacts.

20. The non-transitory computer-readable medium of claim 19, wherein the processor-executable code, when executed by the processing system, further causes the processing system to generate a reconstructed image free of zipper artifacts utilizing the virtual k-space data from a subset of the virtual channels.
